(19) United States
(12) Patent Application Publication
(10) Pub. No.: US 2026/0171262 A1
(43) Pub. Date: Jun. 18, 2026

(54) SCANNING ASSISTANCE SYSTEM AND SCANNING ASSISTANCE METHOD

(71) Applicant: GE Precision Healthcare LLC, Waukesha, WI (US)

(72) Inventors: Feng Li, Beijing (CN); Xinyuan Wang, Beijing (CN); Ziqiang Du, Beijing (CN)

(21) Appl. No.: 19/423,753

(22) Filed: Dec. 17, 2025

(30) Foreign Application Priority Data
Dec. 18, 2024 (CN) 202411867449.8

(51) Int. Cl. G16H 70/20

(52) U.S. Cl. CPC G16H 70/20

(57) ABSTRACT

A scanning assistance system and method is described. The scanning assistance system is connected to an imaging device. The scanning assistance system includes: a classification module, which selects a classifier based on a type of information input in the scanning process, to obtain feature data and question context data corresponding to the information; a decision module, which performs a search in a pre-constructed knowledge base and selects a knowledge base capable of processing the feature data and the question context data; and a control module, which, extracts from a corresponding knowledge base a question corresponding to the feature data and the question context data and a knowledge context for resolving the question, and provides a solution corresponding to the question by using a large language model. A question can be resolved quickly in a scanning process, and a scanning procedure can be optimized.

### CROSS-REFERENCE TO RELATED APPLICATIONS

This application claims priority to Chinese Application No. 202411867449.8, filed on Dec. 18, 2024, the disclosure of which is incorporated herein by reference in its entirety.

### TECHNICAL FIELD

The present application relates to the technical field of medical devices, and in particular, to a scanning assistance system and a scanning assistance method.

### BACKGROUND

At present, medical imaging systems are being used more and more widely to scan subjects (e.g., a human body) to obtain medical images of specified regions to provide useful information for medical diagnosis.

The medical imaging system includes a medical imaging device. The medical imaging device includes a computed tomography (CT) device, a magnetic resonance imaging (MRI) device, a positron emission tomography (PET) device, and a single photon emission computed tomography (SPECT), etc.

Scanning using the medical imaging device is a complex process involving related knowledge in a plurality of fields. A user may encounter various questions in the process of scanning using the medical imaging device, and the user needs to resolve these questions through long-term accumulation and learning.

It should be noted that the above introduction of the background is only for the convenience of clearly and completely describing the technical solutions of the present invention, and for the convenience of understanding for those skilled in the art. The above technical solutions are not considered to be well known to those skilled in the art merely because they are set forth in the Background of the present invention.

### SUMMARY OF THE INVENTION

The inventors found that users encounter various questions in the process of scanning using a medical imaging device. However, the users resolve the questions only through their own knowledge and experience and cannot obtain timely feedback and suggestions. In addition, with the development of technology and clinical applications, the types and number of questions encountered by the users in the process of scanning using the medical imaging device will also increase, and the knowledge and experience of related questions need to be updated at any time, which reduces the efficiency of the users using the medical imaging device, and makes the medical imaging device unable to exert its optimal performance.

In order to resolve at least one of the foregoing questions or other similar questions, embodiments of the present application provide a scanning assistance system and a scanning assistance method. Information inputted in a scanning process is classified to form feature data and/or question context data corresponding to the information, a search is performed in a knowledge base based on the feature data and/or the question context data, and based on a searched corresponding question and a knowledge context for resolving the question, a solution corresponding to the question is provided by using a large language model. Therefore, the question can be resolved quickly, and the scanning procedure can be optimized.

According to an aspect of the embodiments of the present application, a scanning assistance system is provided. The system includes: a classification module, which selects a classifier based on a type of inputted information in a scanning process to process the information, to obtain feature data and/or question context data corresponding to the information; a decision module, which performs a search in a pre-constructed knowledge base based on the feature data and/or the question context data, and selects a knowledge base capable of processing the feature data and/or the question context data; and a control module, which, based on the selection of the decision module, extracts from a corresponding knowledge base a question corresponding to the feature data and/or the question context data and a knowledge context for resolving the question, and provides a solution corresponding to the question by using a large language model.

In some embodiments, the system further comprises a combining module, which combines output results of the large language model to generate a feedback result. In some embodiments, the scanning process comprises at least one of the following: a protocol selection phase, a patient setup phase, a scan prescription phase, a scan acquisition phase, and a post processing phase. In some embodiments, the type of the information in the scanning process comprises at least one of the following: patient information, medical institution information, radiology information, user interaction information, system log, image information, and video information. In some embodiments, the feature data and/or the question context data comprise/comprises at least one of the following: information describing a phenomenon and a feature of the question; information of a function performed; and system configuration information, wherein the system configuration information comprises at least one of the following: hardware information; software information; and production line and type information. In some embodiments, the pre-constructed knowledge base comprises knowledge bases having a plurality of priorities, and the knowledge base of each priority comprises one or a plurality of databases.

In some embodiments, the knowledge bases having the plurality of priorities comprise a knowledge base of a first priority, a knowledge base of a second priority, and a knowledge base of a third priority; the knowledge base of the first priority comprises a feature database and a feature usage database; the knowledge base of the second priority includes an advanced practice database and a network security database; and the knowledge base of the third priority comprises a system analysis database, a troubleshooting database, and a service description database. In some embodiments, the system further includes an update module, which updates the pre-constructed knowledge base based on a predetermined policy and/or a requirement. In some embodiments, the system further includes a scheduling module, which, based on the feedback result, schedules the medical imaging device to perform a corresponding operation.

According to another aspect of the embodiments of the present application, a scanning assistance system is provided and is connected to a medical imaging device. The system includes: a classification module, which selects a classifier based on an inputted medical image to process the medical image, to obtain a feature question and/or question context data corresponding to the medical image, wherein the feature question and/or the question context data are/is a feature question and/or question context data corresponding to a medical image artifact; a decision module, which performs a search in a pre-constructed knowledge base based on feature data and/or the question context data corresponding to the medical image artifact, and selects a knowledge base capable of processing the feature question and/or the question context data corresponding to the medical image artifact; and a control module, which, based on the selection of the decision module, extracts from a corresponding knowledge base a question corresponding to the feature data and/or the question context data and a knowledge context for resolving the question, and provides a solution to the question by using a large language model.

According to still another aspect of the embodiments of the present application, a scanning assistance method is provided and is applied to a medical imaging device. The method includes: selecting a classifier based on a type of inputted information in a scanning process to process the information, to obtain feature data and/or question context data corresponding to the information; performing a search in a pre-constructed knowledge base based on the feature data and/or the question context data, and selecting a knowledge base capable of processing the feature data and/or the question context data; and based on the selection. extracting from a corresponding knowledge base a question corresponding to the feature data and/or the question context data and a knowledge context for resolving the question, and providing a solution corresponding to the question by using a large language model.

One of the beneficial effects of the embodiments of the present application is that, information inputted in a scanning process is classified to form feature data and/or question context data corresponding to the information, a search is performed in a knowledge base based on the feature data and/or the question context data, and based on a searched corresponding question and a knowledge context for resolving the question, a solution corresponding to the question is provided by using a large language model. Therefore, information is classified, features are extracted, and a search is performed in a knowledge base for a question and a method for resolving the question. In this way, the user can be helped to quickly resolve the question encountered in the scanning process, so that the user can use a device more efficiently and optimize the scanning procedure.

With reference to the following description and drawings, specific implementations of the present application are disclosed in detail. It should be understood that the implementations of the present application are not limited in scope thereby. Within the scope of the spirit and clauses of the appended claims, the implementations of the present application comprise many changes, modifications, and equivalents.

The features described and/or illustrated for one implementation may be used in one or more other implementations in the same or similar manner, be combined with features in other embodiments, or replace features in other implementations.

It should be emphasized that the term “include/comprise/have”, when used herein, refers to the presence of features, integrated components, or assemblies, but does not preclude the presence or addition of one or more other features, integrated components, or assemblies.

### BRIEF DESCRIPTION OF THE DRAWINGS

The above and other objects, features and advantages of the embodiments of the present application will become more apparent from the following detailed description with reference to the drawings, in which:

FIG. 1 is a schematic diagram of a scanning assistance system according to an embodiment of the present application;

FIG. 2 is a schematic diagram of a classification module according to an embodiment of the present application;

FIG. 3 is a schematic diagram of a knowledge base according to an embodiment of the present application;

FIG. 4 is a schematic diagram of a control module according to an embodiment of the present application;

FIG. 5 is a schematic flowchart of an operation for a medical image artifact in a scanning assistance system according to an embodiment of the present application;

FIG. 6 is a schematic diagram of a scanning assistance method according to an embodiment of the present application;

FIG. 7 is a schematic diagram of a CT imaging device according to an embodiment of the present application; and

FIG. 8 is a schematic diagram of a CT imaging system according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the present application may be employed are indicated. It should be understood that the present application is not limited to the described implementations, and include all modifications, variations, and equivalents which fall within the scope of the appended claims.

In the embodiments of the present application, the terms “first”, “second”, “upper”, “lower”, etc. are used to distinguish different elements with respect to naming, but do not represent a spatial arrangement, a temporal order, etc. of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one embodiment may be used in one or more other embodiments in an identical or similar manner, combined with features in other embodiments, or replace features in other embodiments.

An embodiment of the present application provides a scanning assistance system, connected to a medical imaging device.

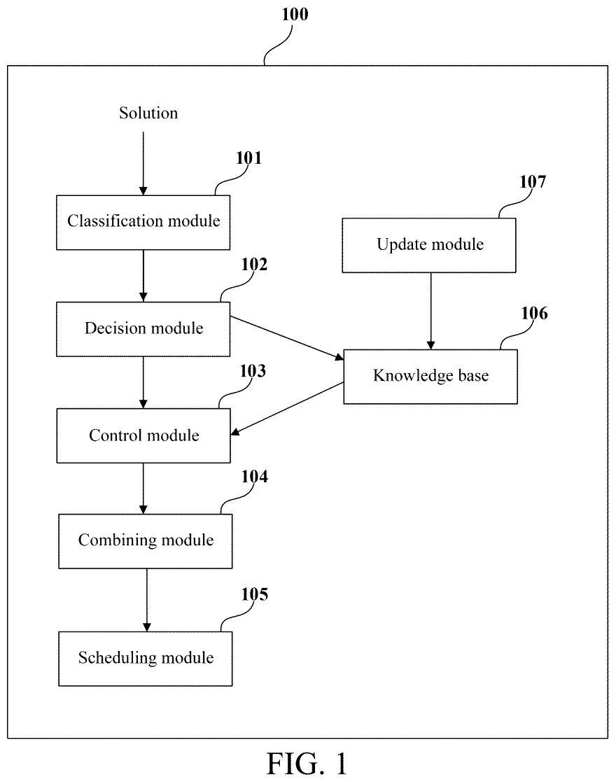

FIG. 1 is a schematic diagram of a scanning assistance system according to an embodiment of the present application. As shown in FIG. 1, the scanning assistance system 100 includes: a classification module 101, which selects a classifier based on a type of inputted information in a scanning process to process the information, to obtain feature data and/or question context data corresponding to the information; a decision module 102, which performs a search in a pre-constructed knowledge base 106 based on the feature data and/or the question context data, and selects a knowledge base capable of processing the feature data and/or the question context data; and a control module 103, which, based on the selection of the decision module 102, extracts from a corresponding knowledge base a question corresponding to the feature data and/or the question context data and a knowledge context for resolving the question, and provides a solution corresponding to the question by using a large language model.

According to the foregoing embodiment, information inputted in the scanning process is classified to form feature data and/or question context (i.e., situational context, background, or situation) data corresponding to the information, a search is performed in a knowledge base based on the feature data and/or the question context data, and based on a searched corresponding question and a knowledge context for resolving the question, a solution corresponding to the question is provided by using a large language model. Therefore, by means of classifying information and extracting features, performing a search in a knowledge base for a question and a prevention for resolving the question, so that the user can be helped to quickly resolve the question encountered in the scanning process, and the user can use the medical imaging device more efficiently and optimize the scanning procedure.

The classification module 101 in this embodiment of the present application is described below.

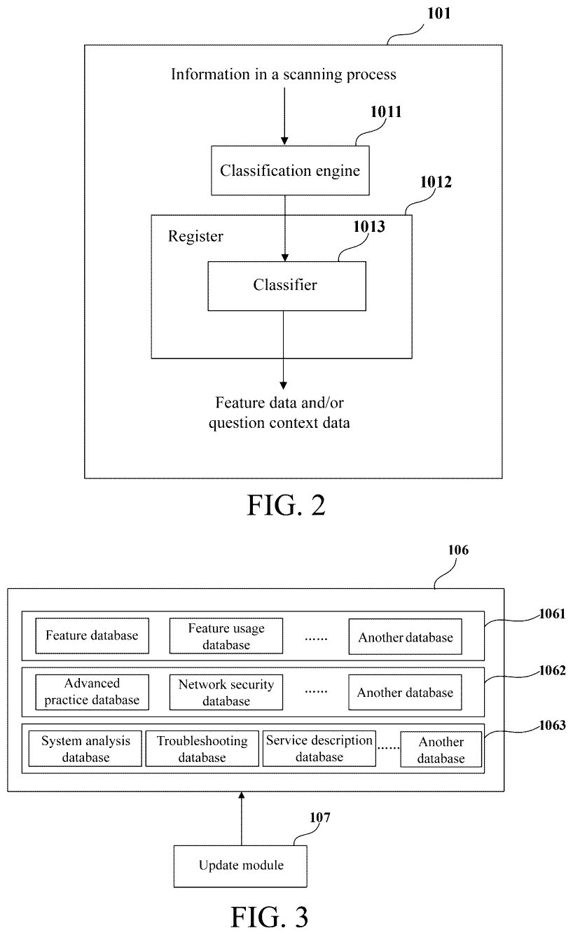

FIG. 2 is a schematic diagram of a classification module 101 according to an embodiment of the present application. As shown in FIG. 2, a classification engine 1011 in the classification module 101 selects a suitable classifier 1013 from a register 1012 based on a type of information in a scanning process, inputs the information in the scanning into the classifier 1013 for processing, and outputs feature data and/or question context data corresponding to the information.

In some embodiments, the scanning process is a process of scanning a patient by a user using a medical scanning device, and includes at least one of the following phases: a protocol selection phase, a patient setup phase, a scan prescription phase, a scan acquisition phase, and a post processing phase. This is not limited in the present application. Information in the scanning process is information generated or used in the foregoing phases.

For example, protocol selection indicates that the user selects different scan protocols and parameters based on different scan needs. A CT scanning device is used as an example. When a brain disease needs to be examined, a cerebral CT scan protocol is selected, or when a lung and heart disease needs to be detected, a thoracic CT scan protocol is selected, and so on. The CT scan device can select different scan modes based on different scan protocols to improve the sensitivity of the scanning. Information in the protocol selection phase is the foregoing scan protocol and/or parameter, and the like.

For another example, patient setup indicates that the user sets basic information such as gender and age of the patient, and may also indicate that factors that may affect the scanning are evaluated and set after the patient's condition is known. For example, when the user has special conditions that may affect the scanning, such as allergies or pregnancy, the user inputs the information to set a suitable scanning scheme to ensure the safety and effectiveness of the scanning. Information in the patient setup phase is the foregoing basic information, setup information, and the like.

For another example, scan prescription indicates that the medical scanning device determines a final scanning scheme based on settings such as protocol selection and patient information, and the user performs confirmation. Information in the scan prescription phase is the foregoing scanning scheme or the like.

For another example, scan acquisition indicates that after the user confirms to start scanning, the medical scanning device starts to scan the patient and acquires data such as images or videos. Information in the scan acquisition phase is the foregoing image, video, or the like.

For another example, post processing indicates that the user performs post processing, such as multi-plane reconstruction and three-dimensional reconstruction, on scanning information collected by the medical scanning device as required, so as to more comprehensively and accurately understand the patient's condition. Information in the post processing phase is information obtained through reconstruction.

In the foregoing embodiment, the scanning process may further include another phase, for example, an image reconstruction phase, or may include a part of the foregoing phases. This is not limited in the present application. Correspondingly, information of another phase is information generated or required in the another phase, and the like. In this embodiment of the present application, the information in the scanning process may be information required or generated in the scanning process, such as the basic information in the patient required in the patient setup phase, or image or video information generated in the scan acquisition phase. In some embodiments, the type of the information in the scanning process includes at least one of the following: patient information, medical institution information, radiology information, user interaction information, system log, image information, and video information.

The patient information may include basic information of the patient, information of a region that needs to be scanned, special information of the patient that may affect scanning, and the like. The medical institution information may include information in a hospital information system (HIS). The hospital information system is a kernel system of hospitals, integrates information in a plurality of aspects such as patient information and medical resources, and can provide comprehensive data support. The radiology information may include information in a radiology information system. The radiology information management system is a procedure management system dedicated to radiology, can manage procedures such as reservation, execution, and reporting of radiological examinations, and stores related information. The user interaction information may include content inputted or clicked by the user on the medical scanning device. For example, the user clicks to select a certain scanning protocol, or the user inputs patient information, and so on. The system log may include operation information, application information, and the like of the medical scanning device. The image information (DICOM images) may include image documents scanned by the medical scanning device. The video information may include a video document obtained by scanning by the medical scanning device.

In the foregoing embodiment, information may be classified into the foregoing types based on the source, use, and format of the information in the scanning process, which is not limited in the present application. The type of the information in the scanning process may further include another type, may include a part of the foregoing types, or a type obtained by further integrating the foregoing types, etc.

In the embodiment of the present application, for the information in the scanning process, different types of information need to be processed by different classifiers by using different algorithm models. For example, the patient information is text information, which needs to be processed by using a text algorithm model, and the video information needs to be processed by using a video algorithm model.

As shown in FIG. 2, the classification engine 1011 classifies information inputted in the scanning process, finds a suitable classifier 1013 from a register based on different types of information, and sends the information in the scanning process to the classifier 1013. The register 1012 is configured to register or store different classifiers, and designate corresponding classifiers for different types of information, to ensure that the classification engine 1011 can use a correct classifier based on the type of information. For example, in the register 1012, a classifier corresponding to image information is designated as a convolution model, and the convolution model may be a residual neural network (ResNet) model. For another example, a classifier corresponding to text information such as patient information and medical institution information is designated as a transformer model.

Therefore, after classifying the information in the scanning process, the classification engine 1011 can directly find the classifier 1013 corresponding to the type of the information in the register 1012, and input the information into the classifier 1013 for processing, and the classifier 1013 processes the information in the scanning process to obtain feature data and/or question context data corresponding to the information.

In some embodiments, the feature data and/or the question context data include at least one of the following: information describing a phenomenon and a feature of the question; information of a function performed; and system configuration information, wherein the system configuration information includes at least one of the following: hardware information; software information; and production line and type information.

The information describing the phenomenon and the feature of the question and the information of the function performed may be obtained by the classification module 101. For the information describing the phenomenon and the feature of the question, an example in which the information inputted in the scanning process is image information is used, the image information is processed by the classifier 1013 selected by the classification engine 1011, and an output result may be that an artifact appears in the image, or the image is overexposed, or more specifically, related data of an artifact of the image information, a specific overexposure parameter of the image information, or the like is outputted.

For the information of the function performed, an example in which the information inputted in the scanning process is the user interaction information and the system log is used. The user interaction information and the system log are separately processed by the classifier 1013 selected by the classification engine 1011, and an output result may be that the user confirms to use a chest scanning protocol for scanning, and the medical imaging device performs chest scanning.

For the system configuration information, the system configuration information may be provided by the medical scanning device itself, or may be obtained by a cloud server. This is not limited in the present application. Furthermore, the system configuration information may further include other information of the medical scanning device itself in addition to the hardware information, the software information, the production line and type information.

In the foregoing embodiment, the feature data and/or the question context data may include all of the foregoing information, or may include a part or one type of the foregoing information. In the embodiment of the present application, as shown in FIG. 1, the decision module 102 performs a search in the pre-constructed knowledge base 106 based on the feature data and/or the question context data, and selects a knowledge base capable of processing the feature data and/or the question context data. The knowledge base in this embodiment of the present application is described below.

FIG. 3 is a schematic diagram of a knowledge base 106 according to an embodiment of the present application.

In some embodiments, the pre-constructed knowledge base includes knowledge bases having a plurality of priorities, and the knowledge base of each priority includes one or a plurality of databases. Sources of the database may include a user manual, a service manual, a system manual, expert experience, and clinical expertise. The plurality of databases pre-construct a knowledge base including a plurality of priorities before the user uses the medical imaging device. In the embodiment of the present application, the database may also have other sources, such as a manual input by the user. This is not limited in the present application. In some embodiments, the knowledge bases of the plurality of priorities include a knowledge base of a first priority, a knowledge base of a second priority, and a knowledge base of a third priority. The knowledge base of the first priority includes a feature database and a feature usage database. The second priority knowledge base includes an advanced practice database and a network security database. The knowledge base of the third priority includes a system analysis knowledge base, a troubleshooting knowledge base, and a service description knowledge base.

As shown in FIG. 3, the pre-constructed knowledge base includes knowledge bases of three priorities: a knowledge base 1061 of a first priority, a knowledge base 1062 of a second priority, and a knowledge base 1063 of a third priority. The knowledge base of each priority includes one or a plurality of databases. FIG. 3 is used as an example. The knowledge base 1061 of the first priority may include a feature database and a feature usage database, and may further include other databases. The knowledge base 1062 of the second priority may include an advanced practice database and a network security database, and may further include other databases. The knowledge base 1063 of the third priority may include a system analysis database, a troubleshooting database, and a service description database, and may further include other databases.

The feature database and the feature usage database may include various feature data and data relating to the feature data. For example, the feature database and the feature usage database may include different feature data of a medical image artifact and possible factors relating to the medical image artifact, or may include all operations that may resolve a medical image artifact. The advanced practice database may include practice operation data relating to advanced features. For example, the advanced practice database may include practice operations relating to an uncommon feature when the feature appears, or may include practice operation data relating to feature data that is not in the database 1061 of the first priority. The network security database may include related data for ensuring the availability and stability of the medical scanning device. The system analysis database may include related analysis data of the medical scanning device. The troubleshooting database may include data relating to possible failures of the medical scanning device. The service description database may include data relating to service manuals in the medical scanning device.

The foregoing classification manner is only an example. According to different scenarios, different scanning processes, different scanning objects, or the like, the foregoing databases may also be classified in other classification manners. For example, the troubleshooting database is used as a database in a knowledge base of a fourth priority that is a higher level. For another example, the network security database is used as a database in a knowledge base of a third priority, and so on.

In the foregoing embodiment, as shown in FIG. 3, when the decision module 102 performs a search in the knowledge base 106, the decision module 102 first performs a search in the knowledge base 1061 of the first priority; if no knowledge base capable of processing the feature data and/or the question context is found in the knowledge base 1061 of the first priority, the decision module 102 further performs a search in the knowledge base 1062 of the second priority; if still no knowledge base capable of processing the feature data and/or the question context is found, the decision module 102 further performs a search in the knowledge base 1063 of the third priority until a knowledge base capable of processing the feature data and/or the question context is found. In addition, when performing a search, the decision module 102 may select one knowledge base, which is not limited in the present application, or may select a plurality of knowledge bases for subsequent operations.

In the foregoing embodiment, a higher priority of the knowledge base indicates a higher usage permission. FIG. 3 is used as an example. Priorities of the knowledge base 1061 of the first priority, the knowledge base 1062 of the second priority, and the knowledge base 1063 of the third priority increase in sequence, and then usage permissions of the knowledge bases of the three priorities also increase in sequence. During use, the user may open permissions of knowledge bases of all priorities and perform a search based on specific requirements.

In the embodiment of the present application, FIG. 3 is used as an example. The knowledge bases of the three priorities are divided based on features and subjects, which is not limited in the present application. The knowledge bases having a plurality of priorities may be set based on different permissions and classification requirements.

Therefore, the knowledge base is classified, so that it is convenient to improve the query performance of the knowledge base and reduce the hardware requirements, and it is also convenient to continuously update the knowledge base. In this way, the knowledge base can be continuously expanded. In some embodiments, as shown in FIG. 1, the scanning assistance system 100 further includes an update module 107. The update module 107 updates the pre-constructed knowledge base based on a predetermined policy and/or a requirement.

As shown in FIG. 3, the update module 107 updates the pre-constructed knowledge base 106. The update module 107 may update the pre-constructed knowledge base 106 based on a predetermined policy, for example, the update module 107 updates the knowledge base 106 at fixed time. The update module 107 may also update the pre-constructed knowledge base 106 based on a requirement. For example, when no knowledge base capable of processing the feature question and/or the question context data is found in the knowledge base 106, the update module 107 updates the knowledge base 106 through searching, or the user manually updates the knowledge base 106 through the update module 107 based on the feature question and/or the question context data. Therefore, by continuously expanding the knowledge base, the scanning assistance system can be gradually improved, so that the scanning assistance system can resolve more questions that occur in the scanning process. The control module in this embodiment of the present application is described below.

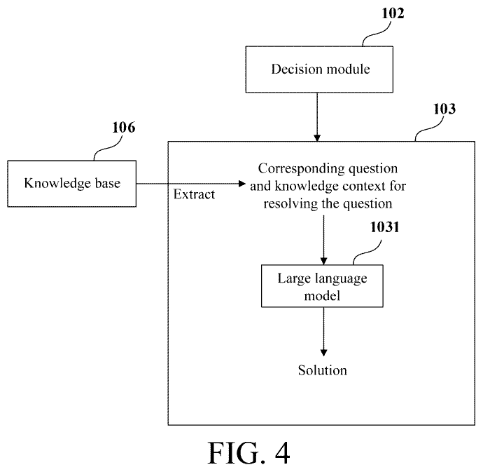

FIG. 4 is a schematic diagram of a control module 103 according to an embodiment of the present application. As shown in FIG. 4, the control module 103, based on the selection of the decision module 102, extracts from the knowledge base 106 a question corresponding to the feature data and/or the question context data and a knowledge context for resolving the question, and processes the corresponding question and the knowledge context for resolving the question by using a large language model (LLM), to obtain a solution corresponding to the question.

For example, a question of the medical image artifact is used as an example. The decision module 102 performs a search in the knowledge base 106 based on related data of the medical image artifact and/or the question context and selects a knowledge base capable of processing the question of the medical image artifact. The control module 103 extracts the corresponding question from the knowledge base, that is, the question of the medical image artifact and a knowledge context capable of resolving the question, and processes the question and the knowledge context corresponding to the question by using the large language model 1031 to obtain a solution corresponding to the question. The solution can make the artifact of the image disappear. In some embodiments, as shown in FIG. 1, the scanning assistance system 100 further includes a combining module 104, which combines output results of the large language model to generate a feedback result.

As shown in FIG. 1 and FIG. 4, the combining module 104 in the scanning assistance system 100 combines output results of the large language models 1031 to generate a feedback result. The feedback result may include a solution for resolving the question, a suggested operation, a question warning, or a possible generated question. In addition, the feedback result may further indicate that the medical scanning device automatically performs a non-critical operation or fills in related information. In some embodiments, as shown in FIG. 1, the scanning assistance system 100 further includes a scheduling module 105, which schedules the medical imaging device to perform a corresponding operation based on the feedback result.

For example, an operation suggestion is displayed to the user on a message window, or the medical imaging device is scheduled to perform a corresponding operation by calling a script or a back-end service, so that the question corresponding to the feature data and/or the question context data can be resolved by prompting the user to perform a related operation or automatically scheduling the medical imaging device. In this way, the scanning is performed smoothly. The operation of the scanning assistance system is described below by using the medical image artifact as an example.

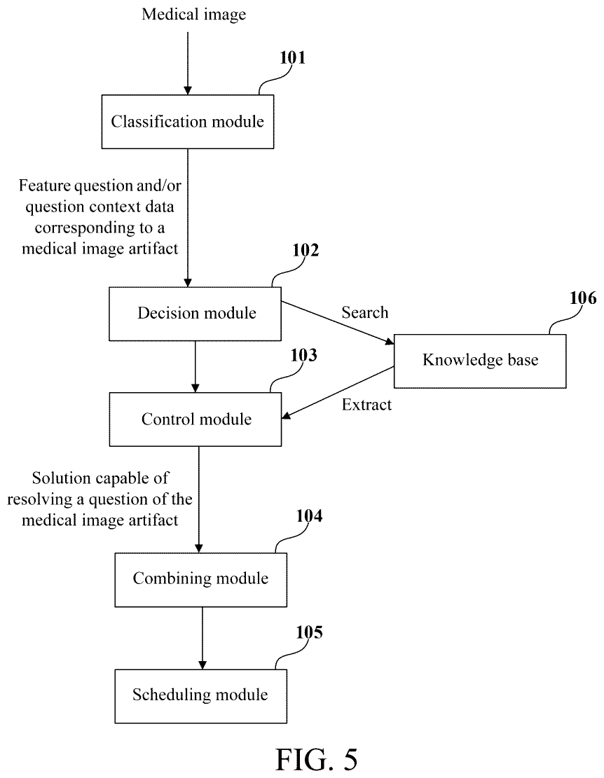

FIG. 5 is a schematic flowchart of an operation for a medical image artifact in a scanning assistance system according to an embodiment of the present application. In some embodiments, the classification module 101 selects a classifier based on an inputted medical image to process the medical image, to obtain a feature question and/or question context data corresponding to the medical image, wherein the feature question and/or the question context data is a feature question and/or question context data corresponding to a medical image artifact.

The decision module 102 performs a search in a pre-constructed knowledge base 106 based on feature data and/or the question context data corresponding to the medical image artifact, and selects a knowledge base capable of processing the feature question and/or the question context data corresponding to the medical image artifact.

The control module 103, based on the selection of the decision module 102, extracts from the corresponding knowledge base 106 a question corresponding to the feature data and/or the question context data and a knowledge context for resolving the question, and provides a solution to the question by using a large language model. The solution can resolve the question of the medical image artifact.

As shown in FIG. 5, the classification module 101 selects a classifier based on an inputted medical image to process the medical image, for example, selects a ResNet model to process the medical image, to obtain a feature question and/or question context data corresponding to the medical image. The feature question and/or the question context data are/is a feature question and/or question context data corresponding to a medical artifact. The medical image artifact represents various forms of imaging that appear on an image even though the scanned object originally does not have the imaging, and the feature question and/or the question context data corresponding to the medical image artifact may represent specific data such as a shape, a density change value, and a scanning parameter of the medical artifact.

The decision module 102 performs a search in a pre-constructed knowledge base 106 based on feature data and/or the question context data corresponding to the medical image artifact, and selects a knowledge base capable of processing the feature question and/or the question context data corresponding to the medical image artifact. A related knowledge base corresponding to each parameter of the medical image artifact may be searched. For example, when the medical image artifact is a radial artifact, a related knowledge base corresponding to the radial artifact is searched, and a knowledge base capable of processing a feature question and/or question context data corresponding to the radial artifact is selected.

The control module 103, based on the selection of the decision module 102, extracts, from the knowledge base 106 capable of processing the feature question and/or the question context data corresponding to the medical image artifact, a question corresponding to the feature question and/or the question context data corresponding to the medical image artifact and a knowledge context for resolving the question, and provides a solution for resolving the question by using a large language model. That is, the control module 103 extracts from the knowledge base 106 selected by the decision module 102 a question corresponding to the medical image artifact and a knowledge context capable of resolving the medical image artifact, and provides a solution by using the large language model. The radial artifact is used as an example. The control module 103 extracts from the knowledge base a question corresponding to the radial artifact and a knowledge context capable of resolving the radial artifact. For example, the question corresponding to the radial example is that there is metal in the patient, and the knowledge context capable of resolving the radial artifact is to remove a metal object or use an algorithm for removing a metal artifact. The control module 103 then uses the large language model to process the question and the knowledge context, and provides a solution to remove the metal object from the patient or use an algorithm for removing the metal artifact. Although not shown in FIG. 5, in some implementations, a database in the knowledge base 106 may be updated by the update module 107. A specific update manner is as described above, and details are not described herein again.

In some embodiments, as shown in FIG. 5, the combining module 104 combines output results of the large language models to generate a feedback result. The foregoing radial artifact is used as an example. The feedback result generated through combination by the combining module 104 based on the output results of the large language model may include: a solution to resolve the radial artifact, a recommended solution, possible questions using each solution, and some other information, such as related information about the metal object.

In some other embodiments, as shown in FIG. 5, the scheduling module 105 can schedule the medical imaging device based on the feedback result to perform a corresponding operation. The radial artifact is used as an example. The scheduling module 105 can display an operation recommendation on the user interface to remove a metal object in the patient's body or use an algorithm for removing the metal artifact, and can also display a recommended solution and other related information described above. If the user selects to use the algorithm for removing the metal artifact, the scheduling module 105 schedules the medical imaging device by using a script or a back-end service to execute the algorithm for removing the metal artifact, so as to perform scanning.

Therefore, the question of artifacts in the medical image is resolved. According to the foregoing embodiments, the question occurring in the scanning can be resolved quickly, and the scanning procedure can be optimized. The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

It can be learned from the above embodiments that information inputted in the scanning process is classified to form feature data and/or question context data corresponding to the information, a search is performed in a knowledge base based on the feature data and/or the question context data, and based on a searched corresponding question and a knowledge context for resolving the question, a solution corresponding to the question is provided by using a large language model. Therefore, information is classified, features are extracted, and a search is performed in a knowledge base for a question and a method for resolving the question. In this way, the user can be helped to quickly resolve the question encountered in the scanning process, so that the user can use a device more efficiently and optimize the scanning procedure. An embodiment of the present application further provides a scanning assistance system, connected to a medical imaging device. The same content as that of the foregoing embodiments is not repeated.

As shown in FIG. 1, the scanning assistance system 100 includes: a classification module 101, which selects a classifier based on an inputted medical image to classify the medical image, to obtain a feature question and/or question context data corresponding to the medical image, wherein the feature question and/or the question context data are/is a feature question and/or question context data corresponding to a medical image artifact; a decision module 102, which performs a search in a pre-constructed knowledge base 106 based on feature data and/or the question context data corresponding to the medical image artifact, and selects a knowledge base capable of processing the feature question and/or the question context data corresponding to the medical image artifact; and a control module 103, which, based on the selection of the decision module 102, extracts from a corresponding knowledge base a question corresponding to the feature data and/or the question context data and a knowledge context for resolving the question, and provides a solution to the question by using a large language model.

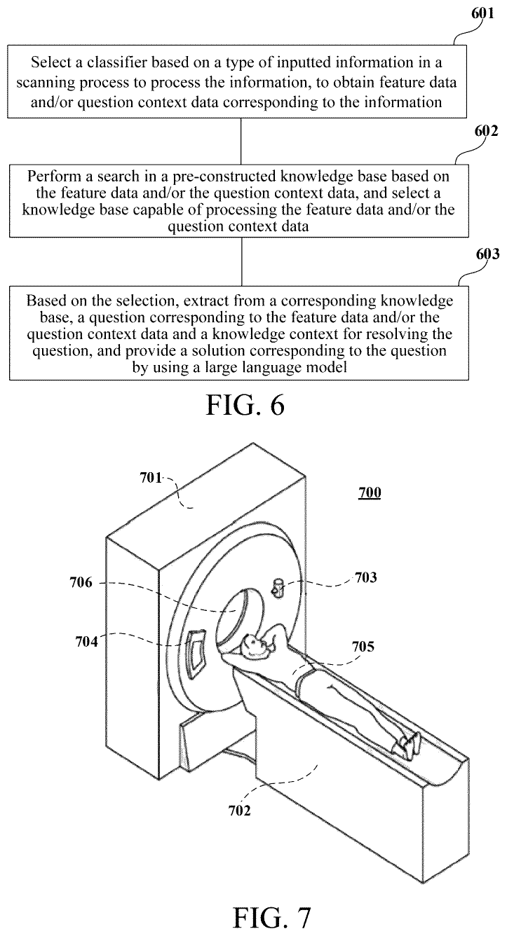

An embodiment of the present application further provides a scanning assistance method. The same content as that of the foregoing embodiments is not repeated. FIG. 6 is a schematic diagram of a scanning assistance method according to an embodiment of the present application. As shown in FIG. 6, the scanning assistance method includes the following steps.

601: Selecting a classifier based on a type of inputted information in a scanning process to classify the information, to obtain feature data and/or question context data corresponding to the information.

602: Performing a search in a pre-constructed knowledge base based on the feature data and/or the question context data, and selecting a knowledge base capable of processing the feature data and/or the question context data.

603: Based on the selection, extracting from a corresponding knowledge base a question corresponding to the feature data and/or the question context data and a knowledge context for resolving the question, and providing a solution corresponding to the question by using a large language model.

It should be noted that FIG. 6 merely schematically illustrates an embodiment of the present application. The present application is not limited thereto. For example, the order of execution between operations may be appropriately adjusted. In addition, some other operations may be added or some operations may be omitted. A person skilled in the art may make appropriate variations based on the described content, rather than being limited to what is set forth in FIG. 6.

It is worth noting that only the steps related to the present application have been described above, but the present application is not limited thereto. The scanning assistance method may further include other steps, and reference may be made to the related art for details of these steps. An embodiment of the present application further provides a medical device system, which includes the scanning assistance system 100 as described in the embodiment of the first aspect, the content of which is incorporated herein.

The following provides an exemplary description of the medical device system. The medical device system described herein, that is, a device and a system that obtain medical imaging data, may be applied to various medical imaging modalities, including, but not limited to, computed tomography (CT) devices, positron emission tomography (PET)-CT, or any other suitable medical imaging device.

The system obtaining the medical imaging data may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud.

For example, the embodiments of the present application are described above in conjunction with an X-ray computed tomography (CT) device. Those skilled in the art would appreciate that the embodiments of the present application can also be applied to other medical imaging devices.

FIG. 7 is a schematic diagram of a CT imaging device according to an embodiment of the present application, and schematically shows a CT imaging device 700. Referring to FIG. 7, the CT imaging device 700 includes a scanning gantry 701 and a patient table 702. The scanning gantry 701 has an X-ray source 703, and the X-ray source 703 projects an X-ray beam toward a detector assembly or collimator 704 on an opposite side of the scanning gantry 701. A subject under examination 705 can lie flat on the patient table 702 and be moved into a scanning gantry opening 706 along with the patient table 702. Medical imaging data of the subject under examination 705 can be obtained through scanning performed by the X-ray source 703.

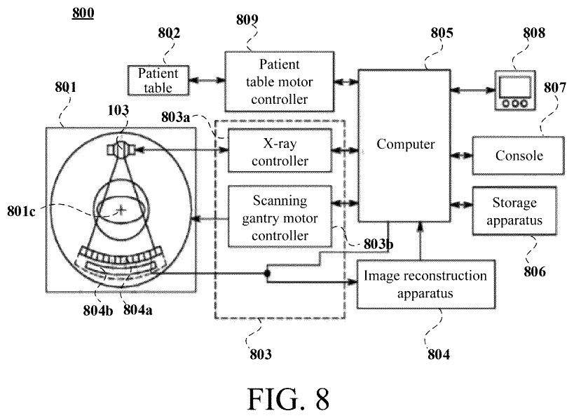

FIG. 8 is a schematic diagram of a CT imaging system according to an embodiment of the present application, and schematically shows a block diagram of a CT imaging system 800. As shown in FIG. 8, the detector assembly 804 includes a plurality of detector units 804a and a data acquisition system (DAS) 804b. The plurality of detector units 804a sense a projected X-ray passing through the subject under examination 705.

The DAS 804b, based on sensing of the detector units 804a, converts collected information into projection data for subsequent processing. During the scanning for acquiring the X-ray projection data, the scanning gantry 801 and components mounted thereon rotate around a center of rotation 801c.

The rotation of the scanning gantry 801 and the operation of the X-ray source 803 are controlled by a control mechanism of the CT imaging system 800. The control mechanism includes an X-ray controller 803a that provides power and a timing signal to the X-ray source 803 and a scanning gantry motor controller 803b that controls the rotational speed and position of the scanning gantry 801. An image reconstruction apparatus 804 receives the projection data from the DAS 804b and performs image reconstruction. A reconstructed image is transmitted as an input to a computer 805, and the computer 805 stores the image in a mass storage apparatus 806.

The computer 805 also receives commands and scanning parameters from an operator through a console 807. The console 807 has an operator interface in a certain form, such as a keyboard, a mouse, a voice activated controller, or any other suitable input apparatus. An associated display 808 allows the operator to observe a reconstructed image and other data from the computer 805. The commands and parameters provided by the operator are used by the computer 805 to provide control signals and information to the DAS 104b, the X-ray controller 803a, and the scanning gantry motor controller 803b. In addition, the computer 805 operates a patient table motor controller 809, which controls the patient table 802 to position the subject under examination 705 and the scanning gantry 801. In particular, the patient table 802 moves the subject under examination 705 to completely or partially pass through the scanning gantry opening 806 in FIG. 8.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SAAS)”. The SAAS may exist between hospitals, between a hospital and an imaging center, or between a hospital and a third-party online diagnosis and treatment service provider.

The embodiments of the present application further provide a non-transitory computer-readable medium, having a computer program stored thereon, where the computer program has at least one code segment, and the at least one code segment is executable by a machine so that the machine performs steps of the method according to the foregoing embodiments. Since the specific implementation of the method has been described in the foregoing embodiments, the contents of which are incorporated herein, no further description is provided herein.

The above method of the present application may be implemented by hardware, or may be implemented by hardware in combination with software. The present application relates to such a computer-readable program that when executed by a logic component, the program enables the logic component to implement the constituent components described above, or enables the logic component to implement various methods or steps as described above. The present application further relates to a storage medium for storing the above program, such as a hard disk, a disk, an optical disk, a DVD, a flash memory, etc.

The method described with reference to the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

1. A scanning assistance system, connected to a medical imaging device, characterized in that the system comprises:
a classification module, which selects a classifier based on a type of information input in a scanning process, to obtain feature data and question context data corresponding to the information;
a decision module, which performs a search in a pre-constructed knowledge base based on the feature data and the question context data, and selects a knowledge base capable of processing the feature data and the question context data; and
a control module, which, based on the selection of the decision module, extracts from a corresponding knowledge base a question corresponding to the feature data and the question context data and a knowledge context for resolving the question, and provides a solution corresponding to the question by using a large language model.

2. The system according to claim 1, wherein the system further includes a combining module, which combines output results of the large language model to generate a feedback result.

3. The system according to claim 1, wherein the system further includes the scanning process comprising at least one of the following: a protocol selection phase, a patient setup phase, a scan prescription phase, a scan acquisition phase, and a post processing phase.

4. The system according to claim 3, wherein, the type of the information in the scanning process comprises at least one of the following: patient information, medical institution information, radiology information, user interaction information, system log, image information, and video information.

5. The system according to claim 1, wherein at least one of the feature data and the question context data includes at least one of the following:
information describing a phenomenon and a feature of the question;
information of a function performed; and
system configuration information, wherein the system configuration information comprises at least one of the following:
hardware information;
software information; and
production line and type information.

6. The system according to claim 1, wherein, the pre-constructed knowledge base comprises knowledge bases having a plurality of priorities, and the knowledge base of each priority comprises one or a plurality of databases.

7. The system according to claim 6, wherein:
the knowledge bases having the plurality of priorities include a knowledge base of a first priority, a knowledge base of a second priority, and a knowledge base of a third priority;
the knowledge base of the first priority includes a feature database and a feature usage database;
the knowledge base of the second priority includes an advanced practice database and a network security database; and
the knowledge base of the third priority includes a system analysis database, a troubleshooting database, and a service description database.

8. The system according to claim 6, wherein the system further includes an update module, which updates the pre-constructed knowledge base based on a predetermined policy and a requirement.

9. The system according to claim 2, wherein the system further includes a scheduling module, which, based on the feedback result, schedules the medical imaging device to perform a corresponding operation.

10. A scanning assistance system, connected to a medical imaging device, characterized in that the system comprises:
a classification module, which selects a classifier based on a medical image input to classify the medical image, to obtain a feature question and question context data corresponding to the medical image, wherein the feature question and the question context data are a feature question and question context data corresponding to a medical image artifact;
a decision module, which performs a search in a pre-constructed knowledge base based on feature data and the question context data corresponding to the medical image artifact, and selects a knowledge base capable of processing the feature question and the question context data corresponding to the medical image artifact; and
a control module, which, based on the selection of the decision module, extracts from a corresponding knowledge base a question corresponding to the feature data and the question context data and a knowledge context for resolving the question, and provides a solution to the question by using a large language model.

11. A scanning assistance method, applied to a medical imaging device, wherein the method comprises:
selecting a classifier based on a type of information input in a scanning process, to obtain feature data and question context data corresponding to the information;
performing a search in a pre-constructed knowledge base based on the feature data and the question context data, and selecting a knowledge base capable of processing the feature data and the question context data; and
based on the selection, extracting from a corresponding knowledge base, a question corresponding to the feature data and the question context data and a knowledge context for resolving the question, and providing a solution corresponding to the question by using a large language model.
